(19) United States
(12) Patent Application Publication
(10) Pub. No.: US 2018/0127813 A1
(43) Pub. Date: May 10, 2018

(54) Nucleic Acid Sequencing using Indicating Polymerases

(71) Applicant: Parallume, Inc., Los Gatos, CA (US)

(72) Inventor: Robert C. Haushalter, Los Gatos, CA (US)

(21) Appl. No.: 15/701,135

(22) Filed: Sep. 11, 2017

Related U.S. Application Data
(60) Provisional application No. 62/385,709, filed on Sep. 9, 2016.

(51) Int. Cl. C12Q 1/6825; C12Q 1/6869; C12N 9/12; C07K 14/405; C07K 14/435; G01N 21/77; G01N 27/49

(52) U.S. Cl. CPC C12Q 1/6825; C12Q 1/6869; C12N 9/1241; C07K 14/405; G01N 2021/7786; G01N 21/77; G01N 27/49; C07K 2319/60; C07K 2319/61; C07K 14/43504

(57) ABSTRACT

A sequencing-by-synthesis or primer extension determination of a nucleic acid sequence is performed by using an indicating polymerase molecule where said indicating polymerase provides a detectable and measurable change when the correct nucleotide is incorporated by the action of the polymerase.

### RELATED APPLICATION

This application claims the benefit of U.S. Provisional Application No. 62/385,709 filed Sep. 9, 2016, the entire disclosure of which is incorporated herein by reference.

### FIELD

This invention relates to techniques, methods, apparatus, reagents and materials which together form a nucleic acid sequencing system that utilizes an indicating polymerase molecule.

### BACKGROUND

The sequencing of nucleic acids, such as deoxyribose nucleic acid (“DNA”) includes determining the order of the nucleotide bases, (e.g., A, C, T and G), along a direction of a nucleic acid strand. The sequence provides detailed molecular level genetic information about the organism. Although many new sequencing technologies have been developed during recent years to sequence DNA more accurately, less expensively and faster than previous techniques, it is still a laborious, expensive and time consuming process to obtain sequencing information. For example, sequencing instruments using clonal amplification in drops or on slide colonies cost $300,000-600,000 and single molecule sequencing instruments cost above $750,000, which does not include the constantly-required stream of very expensive chemicals, reagents and sample preparation protocols. Much of the high cost of these sequencing systems is due to (a) the optical components (microscopes or wave guides) for systems which employ light detection, (b) the custom chip fabrication required for sequencing systems based on electrical detection and (c) the high cost of special labeled chemicals and reagents required in the single molecule-based systems. Widespread use of such valuable sequencing information is greatly hindered by these high costs. Accordingly, there is a great need to develop hardware and reagents that are vastly less expensive and allow the sequencing information to be obtained in a more efficient manner.

Several known sequencing techniques rely on primer extension to sequence the DNA. Primer extension includes a Primer that is in solution or attached to the solid support, a Target that contains the sequence to be determined, dNTP molecules (which will extend the primer and form the synthesized DNA) and a Polymerase molecule. These techniques are often referred to as sequencing-by-synthesis (SBS).

An example of one such primer extension-mediated technique is pyrosequencing. During pyrosequencing, as the primer is undergoing extension, various chemical species are released into the surrounding solution including pyrophosphate (P₂O₇⁴⁻) molecules from the cleavage of the triphosphate moiety associated with the dNTP molecules during strand incorporation and protons (H⁺). By treating the released pyrophosphate ion with a pyrophosphatase enzyme, additional chemical energy can be obtained from this hydrolysis to drive various subsequent chemical reactions. In one case, the pyrophosphate ions are coupled through various chemical species to luciferin, which emits light in proportion to the number of pyrophosphate ions released during primer extension. Therefore, the sequence of the target DNA strand is determined by noting how much light is released upon incorporation of the proper nucleotides.

Another example of DNA sequencing involves electrochemical detection. In this type of sequencing, when the Primer-Target-Polymerase complex (PTP) is undergoing primer extension protons (W) are also released. These protons may be detected using a pH meter to transduce the amount of protons released into an electrical signal. While it is not difficult to detect protons electrochemically, the relatively large distance between the PTP complex and the electrodes may be up to many microns or even millimeters. This large distance between the sample and detector, which affects the diffusion and signal response rates associated with typical pH electrodes, are much slower than techniques where the diffusion distances are shorter. Longer diffusion distances can lead to lower analyte concentrations at the detector and longer, more expensive analysis times.

In these above examples, the signal generated during SBS is not transduced by the polymerase itself but reagents in solution (pyrosequencing example) or a pH-measuring instrument (electrochemical example).

Accordingly, there is a need in the art for a sequencing technique that utilizes a shorter diffusion distance, is easy to use, has inexpensive hardware, uses unlabeled nucleotides and inexpensive reagents and provides a more efficient high throughput screening process.

### SUMMARY

The instant invention describes methods and compositions to sequence DNA one component of which is an indicating polymerase. When sequencing nucleic acids using sequencing-by-synthesis (SBS), primer extension or other methods, all four dNTP (deoxynucleotide triphosphate) molecules are sequentially added one at a time. When the correct dNTP is added, it is incorporated into the DNA strand being synthesized by action of a polymerase and P₂O₇⁴⁻ and H⁺ ions are released into the surrounding solution. Signals from these P₂O₇⁴⁻ and H⁺ ions in solution, or the chemical reaction products of these ions, are then measured chemically, instrumentally or optically to identify which dNTP molecule was incorporated from which the nucleic acid sequence may eventually be determined. Rather than detecting these reaction products remote to the polymerase from which they emanate, the present invention discloses an indicating polymerase molecule which itself detects the incorporation of the correct dNTP. The indicating polymerase has an attached moiety R₁ which, when the correct dNTP is incorporated in the SBS procedure, transforms into R₂. Detection of the change in the physical or chemical properties of the indicating polymerase from R₁ to R₂ may be correlated with the sequence of the nucleic acid being sequenced.

In one illustrative embodiment, a composition for sequencing a nucleic acid by primer extension or SBS that uses an indicating polymerase comprises a suitable buffer; a nucleic acid to be sequenced; at least one dNTP; a priming sequencing; and an indicating polymerase. In some embodiments, the indicating polymerase changes its physical or chemical properties when the correct dNTP is incorporated. In other embodiments, the indicating polymerase moiety R₁ changes its physical or chemical properties and, when the correct dNTP is incorporated, becomes indicating polymerase moiety R₂.

### BRIEF DESCRIPTION OF THE DRAWING

FIG. 1 illustrates the steps by which the indicating polymerase can be used to sequence a nucleic acid comprises the nucleic acid to be sequenced (S₁), a primer sequence (PS) and the indicating polymerase (P) with attached reporter (R₁) which assemble into the tripartite entity (indicating polymerase, primer sequence and sequence to be determined). When the correct dNTP is incorporated, the R₁ reporter moiety on the indicating polymerase changes to R₂ thereby sensing and indicating the successful incorporation of the dNTP.

FIGS. 2A-2C illustrate one exemplary embodiment of the invention, wherein changes in the current-voltage behavior (cyclic voltammogram FIG. 2A) of the indicating polymerase shows the transformation of R₁ into detectable R₂ which may be used to sequence the nucleic acid. The R₁ moiety displays a certain oxidation and reduction potential (FIG. 2A) which, upon incorporation of the correct dNTP, transforms into R₂. Examples of two different and detectable oxidation-reduction potentials possible for R₂ are shown in FIGS. 2B and 2C.

### DETAILED DESCRIPTION

To address the current limitations discussed above, disclosed herein are compositions and methods that include a system where the chemical sensor that detects the sequencing reaction the polymerase enzyme itself that is performing the primer extension. The polymerase enzyme, which detects the primer extension by changing its physical or chemical properties upon and concomitant with incorporation of the correct dNTP during SBS and primer extension, is called an indicating polymerase. As described above, all known sequencing systems have the sequencing-detecting sensor or reagents external to and physically separated from the sequencing reactions. By eliminating the optical components, external transducing sensors and highly specialized labeled reagents, a high throughput sequencing instrument may be built, using standard, commercially available components and unlabeled nucleotide reagents, which is at least 100 times less expensive than current sequencing instruments.

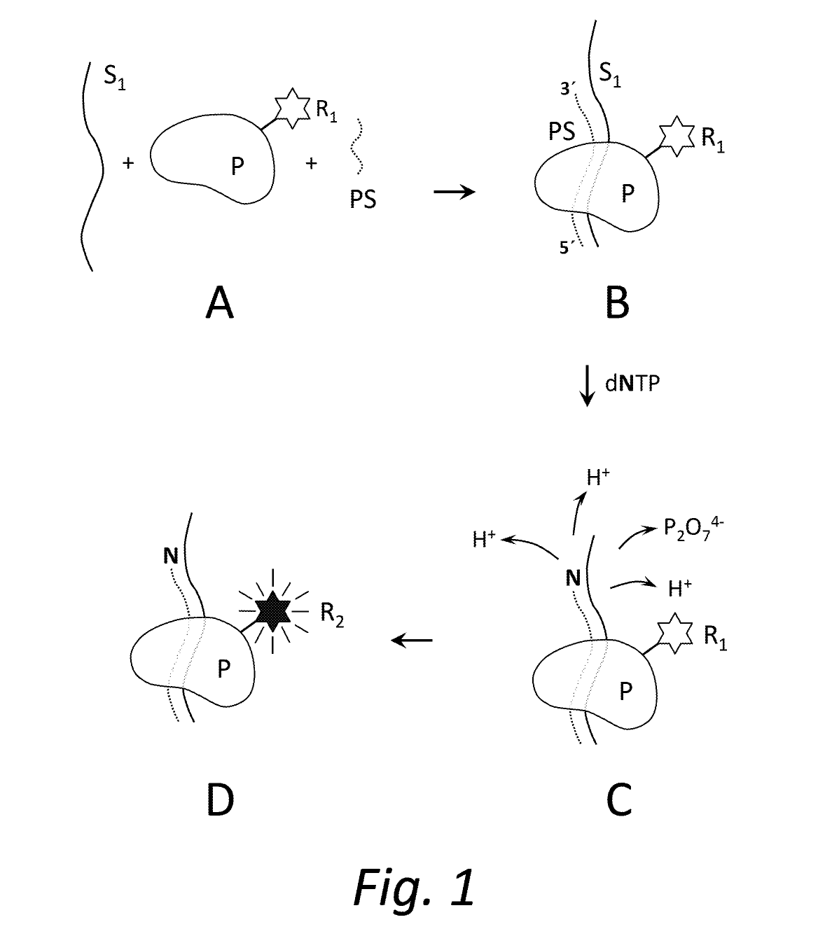

Referring to FIG. 1, for a sequencing-by-synthesis (SBS) or primer extension sequencing method or protocol using the indicating polymerase, the minimum necessary composition comprises a nucleic acid whose sequence S₁ is to be determined, a priming sequence PS and a nucleic acid polymerase protein P. When dNTP (deoxynucleoside triphosphate) molecules, such as deoxyadenosine triphosphate (dATP), deoxyguanosine triphosphate (dGTP), deoxycytidine triphosphate (dCTP), deoxythymidine triphosphate (dTTP) and deoxyuridine triphosphate (dUTP)) molecules are added one at a time to the tripartite S₁—PS—P species, and the correct base is incorporated onto the 3′-end of the extending PS, protons and pyrophosphate ions are released in step C. Detection of these released ions form the basis of pyrosequencing and Ion Torrent®-type sequencing protocols by detecting pyrophosphate and protons, respectively. In these two protocols (i.e., pyrosequencing and Ion Torrent-type), the sensor or signal transducer is either in solution or tens of thousands of molecular diameters away (e.g., sample to electrode distance in electrochemical Ion Torrent-type sequencers. The relatively large molecular distances involved lead to longer sample-detector distance with concomitant increased analysis time, more dilute samples from diffusion effects and a larger sequencing apparatus. Therefore, there is a great need for simpler sequencing methods, smaller samples, shorter sample to detector distances and size reduction for sequencing apparatus.

The smallest and fastest possible sequencing method or protocol would comprise only the three essential S₁, PS and P components (steps A and B of FIG. 1). For example, but not limitation, the sequencing method comprises only S₁, PS and P where the reporter or signal transducing moiety that detects the dNTP incorporation is directly bonded to the polymerase by covalent, electrostatic, hydrophobic-hydrophobic, hydrophilic-hydrophilic, other bonding interactions or combinations thereof. Since the dNTP is incorporated into the extending primer directly upon the surface of the polymerase, and the transducing agent R₁ is bound directly to polymerase, the ions to be detected step C have only a very short distance to travel from creation to detection. It is important to note that the smaller the volume into which the ions are released and detected, the higher the concentration of those ions will be thereby resulting in more sensitive/accurate and faster measurement of the key SBS sequencing events.

All SBS methods for sequencing nucleic acids detect the incorporation of the correct dNTP into the extending primer by measuring a change in some characteristic property that indicates when the correct dNTP is provided but the property does not change when presented with an incorrect dNTP. In the case of pyrosequencing, the energy released from the pyrophosphate hydrolysis by an added pyrophosphatase enzyme is converted to light emitted via luciferase which may be correlated with correct dNTP incorporation. When using an electrochemical sequencing method such as Ion Torrent, the protons released when the correct dNTP is incorporated are measured with a pH electrode. Haushalter previously taught that the protons released may be detected with a pH-sensitive fluorogenic dye molecule, which, in one embodiment, is attached to a bead along with the nucleic acid being sequenced, which is non-fluorescent at higher pH but fluorescent at lower pH.

In the nucleic acid sequencing method of the present invention, the incorporation of the correct dNTP induces a change in the polymerase molecule itself mediating the primer extension. As illustrated in FIG. 1, when the correct dNTP is incorporated, the reporter group or entity R₁ associated with the polymerase molecule changes to R₂ where R₁ and R₂ are distinguishable by some chemical or physical property. This change in the polymerase molecule (R₁→R₂) may be correlated with dNTP incorporation and therefore provide a means of sequencing S₁.

The R₁ reporter group is attached to, bonded to or otherwise intimately associated with the polymerase. The R₁ group may be attached to or associated with the polymerase after the polymerase molecule has been prepared or is attached as the protein is being expressed during synthesis or a combination thereof. The R₁ group may be attached to or associated with the polymerase by means of a covalent bond, ionic bond, hydrophobic-hydrophobic or hydrophilic-hydrophilic interactions, van der Waals, magnetic interactions or any other type of bonding or associative interaction or combinations thereof.

The polymerase may be designed, synthesized or modified by many different means in order to detect the dNTP sequencing reaction including chemical and physical means. Some possible R₁ materials are listed in Table 1 where the possibilities discussed are for illustrative purposes only and are not meant to limit the scope of the invention in any way. The protons and pyrophosphate anions released during correct dNTP incorporation may react directly with R₁ converting R₁ into detectable R₂. Alternatively, the protons and pyrophosphate ions may react with another molecule or entity (not R₁), which may be attached to the polymerase, the priming sequence PS or the nucleic acid being sequenced S₁, as illustrated in FIG. 1, or located nearby in solution, which in turn reacts with R₁ to convert R₁ into the detectable R₂. For example, the released protons could react with a species on the primer P or nucleic acid sequence S₁ which would in turn react with R₁ to convert R₁ into R₂.

In one illustrative embodiment, R₁ is a fluorogenic dye covalently bonded to the polymerase that is colorless at higher pH but turns fluorescent when the protons are released and the pH becomes lowered. When the dye becomes fluorescent against a dark background, the amount of light released from the fluorophore indicates correct dNTP incorporation.

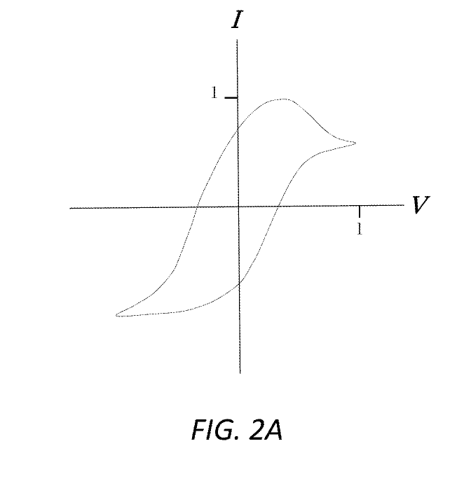

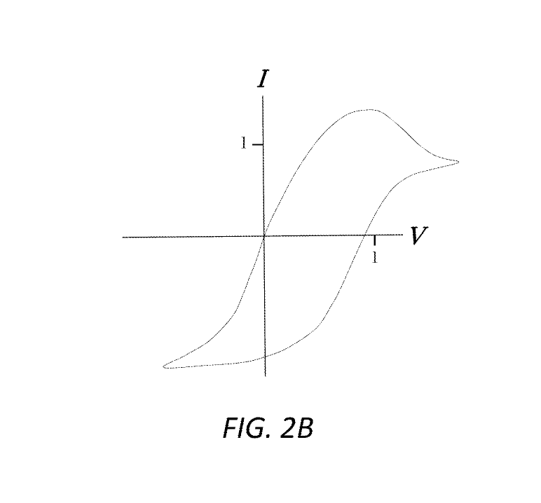

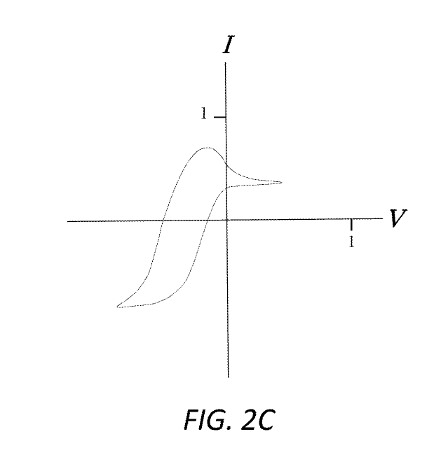

TABLE 1
Types of R₁ to R₂ transformations where R₁ changes to R₂ only upon
correct dNTP incorporation.

| | Change of R₁ to R₂ with correct | |
|---|---|---|
| Types of R₁ | dNTP incorporation | Example of detection methods |
| R₁ is a fluorogenic dye | R₁ changes from non-fluorescent | Optical fluorescence |
| | to fluorescent R₂ upon lowering | measurement |
| | pH or upon reaction with H⁺ or | |
| | P₂O₇⁴⁻ | |
| R₁ is an entity that may | R₁ and R₂ have different and | Measure current (I), voltage (V), |
| change its electrochemical | distinguishable electrochemical | impedance, inductance, |
| oxidation or reduction | oxidation or reduction potentials | capacitance, polarization; |
| potential | (FIG. 2) upon reaction with H⁺ or | measure electrical properties |
| | P₂O₇⁴⁻ | with electrodes or Scanning |
| | | Tunneling Microscope |
| R₁ is an entity that changes | R₁ and R₂ have different and | Observe IR or Raman spectra |
| its vibrational spectrum | distinguishable Infrared (IR) or | |
| | Raman vibrational absorption or | |
| | emission bands that appear or | |
| | disappear upon reaction with H⁺ | |
| | or P₂O₇⁴⁻ | |
| R₁ is an entity that changes | R₁ and R₂ have different and | Measure VIS or UV absorption |
| its color or molar | distinguishable absorption or | or emission spectra |
| absorptivity | emission spectrum for visible or | |
| | ultraviolet wavelengths upon | |
| | reaction with H⁺ or P₂O₇⁴⁻ | |
| R₁ is an entity that can | R₁ and R₂ have different | Determine conformational |
| change its conformation or | conformations upon reaction with | change with Atomic Force |
| shape | H⁺ or P₂O₇⁴⁻ | Microscopy (AFM) |
| R₁ is an entity that changes | R₁ and R₂ have different | Measure magnetic properties |
| its magnetic properties | magnetic properties or number of | with magnetic susceptibility or |
| | unpaired electrons upon reaction | Electron Spin Resonance (ESR) |
| | with H⁺ or P₂O₇⁴⁻ | |
| R₁ is an entity that can | R₁ and R₂ have different and | Measure reflectivity of sample at |
| change its reflectivity | distinguishable reflectivity upon | a given wavelength |
| | reaction with H⁺ or P₂O₇⁴⁻ | |

Since the instant sequencing method of FIG. 1 requires only the molecular S—PS—P components, it is particularly well suited to single molecule sequencing protocols. For single molecule sequencing, it is necessary to rapidly and reliably differentiate the individual strands of nucleic acid to be sequenced. The individual strands are identified by either (a) knowing their fixed location or (b) encoding each strand with an identifier (such as an optical code created from organic dyes, quantum dots or lanthanide materials or mixtures thereof).

In yet another illustrative embodiment, the sequence-indicating (R₁→R₂) transformation could involve electrochemical detection of R₁ modified by reaction with the protons and pyrophosphate ions. This transformation could be measured by measuring the change in conductivity, capacitance, resistance, inductance, voltage, current or combinations thereof when R₁ converts into R₂. As illustrated for example, but not limitation in FIGS. 2A-2C, a cyclic voltammogram of R₁ and R₂ with appropriately configured working, counter and reference electrodes (or, alternatively, with just a working and counter electrode) may be obtained and the differences between R₁ and R₂ voltammograms indicate correct incorporation of the correct dNTP.

In FIGS. 2A-2C, differences between the current and voltages influenced by the R₁ and R₂ transformation are illustrated. Therefore, the electrochemical properties of R₁ and R₂ are different.

In some embodiments, R₁ may not necessarily transform from a direct reaction with the protons or pyrophosphate ions but could be transformed into R₂ by a mediator or transfer molecule which reacts directly itself with the protons or pyrophosphate ions and then subsequently reacts with R₁ to transform R₁ into R₂.

In still another embodiment, when R₁ reacts with the protons or pyrophosphate ions (or the mediator molecule which reacts initially with the protons and pyrophosphate ions, subsequently reacts with R₁), then changes in the emission or absorption bands of the vibrational spectrum of R₁, such as infrared, Raman or other vibrational measurement techniques, will indicate the correct incorporation of a dNTP.

In a further embodiment, upon reaction with protons or pyrophosphate ions, R₁ changes to R₂ with a concomitant change in the wavelength or molar absorptivity of a visible or ultraviolet absorption of emission property thereby indicating the incorporation of a correct dNTP in the SBS sequencing protocol.

It should be noted that it would also be possible to use the heat released upon correct dNTP incorporation to drive the R₁→R₂ transformation instead of the pyrophosphate and protons. Detection of this heat could be combined with the proton and pyrophosphate reactions to detect correct dNTP incorporation.

One should not construe these embodiments, or the embodiments in Table I, as limiting the scope of the invention and many other types of R₁ to R₂, as well as R₁ to R₂ transformations and detection schema are possible.

### EXAMPLES

### Example 1 Indicating Polymerase from Gene Expression

The polymerase proteins used for sequencing are often expressed in hosts such as bacteria, viruses or other cells or organisms. In order to express an indicating polymerase which can be used for SBS or other primer extension methods, a gene to synthesize fluorophores such as Phycoerythrin (PE) or Green Fluorescent Protein (GFP) is inserted into the host gene so that when the polymerase is expressed the PE or GFP is also expressed. These GFP or PE examples represent R₁ in FIG. 1. The DNA sequences needed to express the fluorophore and polymerase may be contiguous or have another sequence inserted between them. Therefore, when the polymerase is expressed, the fluorophore is also expressed and is attached to the polymerase.

Next, the polymerase-fluorophore moiety is provided with a nucleic acid sequence to be determined and a priming sequence suitable for SBS or primer extension. After measuring the fluorophore under non-acidic conditions, the different dNTP molecules are sequentially added and when the correct dNTP is added, the fluorophore R₁ transforms into R₂ which has a different absorption, emission and fluorescence spectrum than R₁. R₂ is therefore distinguished from R₁ and this information is used to determine which dNTP were incorporated (i.e., sequencing the nucleic acid).

### Example 2 Indicating Polymerase from Chemically Modifying a Polymerase

A polymerase expressed in a bacterium is combined with a fluorogenic organic dye like pHrodo® from Life Technologies which is available as a succinimidyl ester and is non-fluorescent at pH=10 but strongly florescent at pH=≤7. The pHrodo succinimidyl ester reacts with groups on the surface of the polymerase protein and covalently binds the fluorophore to the polymerase.

Next, the polymerase-fluorophore moiety is provided with a nucleic acid sequence to be determined and a priming sequence suitable for SBS or primer extension. After setting the fluorogenic pHrodo to its non-fluorescent state R₁, as illustrated in FIG. 1, the different dNTP molecules are sequentially added and when the correct dNTP is added, and the protons and pyrophosphate ions are released, the fluorogenic R₁ transforms into R₂ which has a different absorption, emission and fluorescence spectrum than R₁. R₂ is therefore distinguished from R₁ and this information is used to determine which dNTP were incorporated (i.e., sequencing the nucleic acid).

### Example 3 Indicating Polymerase Using Electrochemical Detection

A reporter molecule R₁ as illustrated in FIG. 1, which has a different oxidation potential and a different cyclic voltammogram when it is in its protonated and unprotonated forms, is attached to the polymerase. This polymerase is now an indicating polymerase that indicates if the correct dNTP has been incorporated by a change in the electrical properties or oxidation/reduction potential of the reporter. This reporter molecule is attached to the polymerase by binding to the surface of the polymerase via hydrophobic and hydrophilic interactions.

After setting the reporter molecule R₁ to its unprotonated state, the different dNTP molecules are sequentially added and, when the correct dNTP is added, protons and pyrophosphate ions are released in step C of FIG. 1. When R₁ reacts with a released proton (i.e., when the correct dNTP is added), its oxidation potential is changed and when the voltage is swept with respect to current and time, R₁ and R₂ give different cyclic voltammograms as illustrated in FIGS. 2A-2C, and this difference can be used to sequence a nucleic acid.

It should be understood that the invention is not limited to the embodiments illustrated and described herein. Rather, the appended claims should be construed broadly to include other variants and embodiments of the invention, which may be made by those skilled in the art without departing from the scope and range of equivalents of the invention. It is indeed intended that the scope of the invention should be determined by proper interpretation and construction of the appended claims and their legal equivalents, as understood by those of skill in the art relying upon the disclosure in this specification and the attached drawings.

What is claimed is:

1. A composition for sequencing a nucleic acid by primer extension or SBS that uses an indicating polymerase comprising:
a) a suitable buffer;
b) a nucleic acid to be sequenced;
c) at least one dNTP;
d) a priming sequencing; and
e) an indicating polymerase which changes its physical or chemical properties when the correct dNTP is incorporated.

2. The composition as in claim 1 where the indicating polymerase changes its absorption, emission, reflective or fluorescent optical properties.

3. The composition as in claim 1 where the indicating polymerase changes its absorption, emission or fluorescent optical properties of a fluorogenic indicator.

4. The composition as in claim 1 where the indicating polymerase changes its shape or conformation.

5. The composition as in claim 1 where the indicating polymerase changes its temperature

6. The composition as in claim 1 where the indicating polymerase changes its vibrational absorption or emission optical properties.

7. The composition as in claim 1 where the indicating polymerase changes its electrical properties, such as oxidation/reduction potential, conductivity, resistivity or impedance

8. The composition as in claim 7 where the indicating polymerase changes are measured with electrodes, a scanning probe tip or by impedance.

9. A composition for sequencing a nucleic acid by primer extension that uses an indicating polymerase comprising:
a) a suitable buffer;
b) a nucleic acid to be sequenced;
c) at least one dNTP;
d) a priming sequencing; and
e) an indicating polymerase where indicating polymerase moiety R₁ changes its physical or chemical properties and, when the correct dNTP is incorporated, becomes indicating polymerase moiety R₂.

10. The composition as in claim 9 where R₁ and R₂ are attached to the polymerase by means of a covalent bond, ionic bond, hydrophobic-hydrophobic or hydrophilic-hydrophilic interactions, van der Waals, magnetic interactions or any other type of bonding or associative interaction or combinations thereof.

11. The composition as in claim 9 where R₁ is a fluorogenic dye in its non-fluorescent state and R₂ is a fluorogenic dye in its fluorescent state.

12. The composition as in claim 9 where the change in physical or chemical property measured for R₁ to R₂ transformation is one or more of the electrical, optical, magnetic, vibrational or thermal properties or combinations thereof of the indicating polymerase.

13. A method for sequencing a nucleic acid using an indicating polymerase comprising:
a) providing a suitable buffer;
b) adding a nucleic acid to be sequenced and a priming sequence;
c) configuring the nucleic acid priming sequence, nucleic acid sequence to be determined and the indicating polymerase to perform primer extension and sequencing by synthesis;
d) adding dNTP with one nucleotide at a time;
e) observing the indicating polymerase change its physical or chemical properties when the correct dNTP is incorporated;
f) correlating the change in the physical or chemical properties of the indicating polymerase with the dNTP added to obtain the nucleic acid sequence.

14. The method as in claim 13 where the change in physical or chemical property measured for R₁ to R₂ transformation is one or more of the electrical, optical, magnetic, vibrational or thermal properties or combinations thereof of the indicating polymerase.

15. The method as in claim 13 where R₁ and R₂ are attached to the polymerase by means of a covalent bond, ionic bond, hydrophobic-hydrophobic or hydrophilic-hydrophilic interactions, van der Waals, magnetic interactions or any other type of bonding or associative interaction or combinations thereof.
